(19) United States
(12) Patent Application Publication
(10) Pub. No.: US 2024/0415149 A1
(43) Pub. Date: Dec. 19, 2024

(54) FEED OR FOOD INGREDIENT DERIVED FROM FIBRE-RICH BIOMASS OF SOY HULLS

(71) Applicant: HAMLET PROTEIN A/S, Horsens (DK)

(72) Inventors: Laila THIRUP, Horsens (DK); Christine BRØKNER, Horsens (DK); Jonatan A. DICKOW, Horsens (DK); Pernille Toft RASMUSSEN, Horsens (DK)

(73) Assignee: HAMLET PROTEIN A/S, Horsens (DK)

(21) Appl. No.: 18/029,775

(22) PCT Filed: Apr. 29, 2021
(86) PCT No.: PCT/EP2021/061281

§ 371 Date: Mar. 31, 2023

(30) Foreign Application Priority Data
Oct. 2, 2020 (EP) 20199889.5

(51) Int. Cl. A23K 10/12; A23K 10/14; A23K 10/16; A23K 10/37; A23K 20/163; A23K 50/10; A23K 50/30; A23K 50/60; A23K 50/75

(52) U.S. Cl. CPC A23K 10/12; A23K 10/14; A23K 10/16; A23K 10/37; A23K 20/163; A23K 50/10; A23K 50/30; A23K 50/60; A23K 50/75

(57) ABSTRACT

The invention provides a feed or food ingredient derived from soy hulls, wherein the feed or food ingredient comprises dietary fibres from the soy hulls in the form of soluble and insoluble polysaccharides, and wherein the dietary fibres from the soy hulls have been partly degraded by one or more carbohydrase(s) selected from mannanase(s), pectinase(s), xylanase(s), glucanase(s), and cellulase(s), into oligosaccharides having from 3 to 30 monomer sugar units (Oligosaccharides DP 3-30), and wherein the feed or food product comprises 5% by weight mono sugars, or less, preferably providing prebiotic effect.

### CROSS-REFERENCE TO RELATED APPLICATIONS

The present application is the U.S. National Stage of International Application No. PCT/EP2021/061281, filed Apr. 29, 2021, and claims priority to European Patent Application No. 20199889.5, filed Oct. 2, 2020.

### FIELD OF THE INVENTION

The present invention relates to a feed or food ingredient derived from a fibre-rich plant biomass of soy hulls, a method for the product of the feed/food ingredient, a use of the feed/food ingredient, and a feed or food product comprising the feed/food ingredient.

### BACKGROUND OF THE INVENTION

Fibre is increasingly important in alternative feeding of production animals. Previously, fibre has been regarded as an anti-nutritional factor that reduces the energy concentration and digestibility of feed, leading to poorer animal performance. Increasing evidence during the last two to three decades has, however, shown that a certain fibre inclusion is beneficial for performance and development of a healthy gut of the animal; Agyekum & Nyachoti (2017), Molist et al (2009). Both the content of soluble and insoluble non-starch polysaccharides (NSP) as well as the content of prebiotic oligosaccharides are part of the dietary fibre which can have positive effects in the animal. More generally, fibre is now investigated intensively to be one of the solutions to restrictions in use of antibiotics and zinc in the animal sector. With less use of antibiotics and zinc, bad performance, disease, and diarrhoea will increase, and it is a serious problem.

Dedicated fibre sources are increasingly used in monogastric feeding. Soy hulls is such a dedicated fibre source, high in total and in insoluble fibres. Surprisingly, it has now been found by the present inventors, that when one treats the fibre biomass soy hulls with specific carbohydrate targeting enzymes, the fibres can release a rather large amount of oligosaccharides, compared to many other natural biomasses that are high in fibre content. Depending on the type of targeting enzymes applied, the nature of oligosaccharides released from the soy hulls and the other biomasses can be different.

In some animal feeding, it is now common to add “exogenous” enzymes to the final diet formulation, but the enzyme is not in action before it is consumed with the rest of the diet, working in the gut environment of the animal (Kiarie et al 2013, Scapini et al 2018). The use of carbohydrase enzymes as a production aid to increase the prebiotic oligosaccharide content of feed biomasses has only been described to a limited degree. The potential effect of some oligosaccharides is expected to be prebiotic, when the oligosaccharide-enriched source is fed to production animals, as for example pigs and poultry, resulting in increased animal performance and health. For human food products, or research purposes, biomasses have been treated with enzymes and/or physical/chemical means to produce, extract and purify prebiotic oligosaccharides. Alternatively, some prebiotic products are chemically synthesized and purified (for example polydextrose, dextrin) for the same use. This type of products are expensive and, therefore, not used to a significant degree in animal feed; Babber et al (2015); Aachary & Prapulla (2011); Dotsenko et al (2017); Kurakake et al (2006). At present, it appears that there are no feed or food products on the market originating from soy hulls by carbohydrase enzyme treatment, having increased specialised oligosaccharide content in comparison with the untreated soy hulls. There is neither any prior art that describes a carbohydrase enzyme treated soy hull for consume; some literature only describe that soy hulls can be added to the diet together with an external carbohydrase enzyme source before feeding of the animal.

The present inventors have tested different soy hulls products treated with carbohydrate targeting enzymes, such as mannanase, xylanase, or pectinase, in piglet feeding trials and in vitro piglet trials, and found improved performance compared to a control group.

The object of the present invention is to provide a new feed or food ingredient having a distinctive composition, that originates from a fibre-rich plant biomass of soy hulls.

Another object is to provide, on a favourable cost basis for the applied carbohydrate targeting enzymes, a new improved feed or food ingredient originating from soy hulls, which source comprises both a certain amount of complex carbohydrates in the form of dietary fibres and an increased amount of prebiotic oligosaccharides derived by action of carbohydrate targeting enzymes, which is advantageous when used in an animal or human diet.

Yet another object is to provide a new feed or food ingredient obtainable by treatment of soy hulls with carbohydrate targeting enzymes.

Furthermore, it is an object to provide an improved feed or food ingredient containing prebiotic oligosaccharides, having higher digestibility in comparison with the untreated (raw) soy hulls.

Finally, it is an object to provide a new product with high water binding capacity and low viscosity; both these properties are important for the product's application in an animal diet.

These objects are fulfilled with the product of the present invention.

### SUMMARY OF THE INVENTION

Accordingly, in a first aspect the present invention relates to a feed or food ingredient derived from soy hulls, wherein the feed or food ingredient comprises dietary fibres from soy hulls in the form of soluble and insoluble polysaccharides, and wherein the dietary fibres from the soy hulls have been partly degraded by one or more carbohydrase(s) selected from mannanase(s), pectinase(s), xylanase(s), glucanase(s), and cellulase(s), into oligosaccharides having from 3 to 30 monomer sugar units (Oligosaccharides DP 3-30), and wherein the feed or food product comprises 5% by weight monosaccharides, or less.

In the context of the present invention, the oligosaccharides DP 3-30 derived from the dietary fibres do not comprise raffinose, stachyose, and verbascose.

Surprisingly, the present inventors have found that when they treated a range of different fibre-rich plant biomasses with selected carbohydrases, viz. enzymes acting on complex polysaccharides, soy hulls stood out as a biomass from which a quite large amount of oligosaccharides can be released. Also, a number of selected mannanases, pectinases, xylanases, cellulases, and β-glucanases produce a significant amount of oligosaccharides from soy hulls, in comparison with what is typically seen from other biomasses. It is expected that some of these oligosaccharides can act as prebiotics in animal feed or human food, depending on the chemical characteristics of the oligosaccharides.

It is further surprising that such products when they are applied in the animal diet can increase animal performance, such as piglet and poultry performance. The inventors have confirmed this in piglet feeding trials. The known mode of actions lying behind the increased performance when one uses prebiotic oligosaccharides are: improving/stabilizing a healthy microbial community in the gut, reduction of the risk of intestinal infection and inflammation, improvement of intestinal functions (stool bulking, regulatority and consistency), improvement of intestinal barrier function, regulation/modulation of immune functions and modulation of gastro-intestinal peptide production and energy metabolism; Roberfroid et al (2010). The best known and best studied and documented oligosaccharides are fructo-oligosaccharides (FOS); Ghoddusi et al (2007); Probert et al (2004), typically derived from inulin; and mannan-oligosaccharides (MOS); Corrigan et al (2015); Zivkovic et al (2011); Kim et al (2010). In the case of MOS, it is the type of MOS derived from yeast cell walls that are well documented for their effect. They are α-1,3 and α-1,6 branched mannans. Examples of new up-coming groups of prebiotic oligosaccharides are: xylo-oligosaccharides (XOS); Liu et al (2018); Moura et al (2008); Aachary & Prapulla (2011); Dotsenko et al (2017); Nielsen et al (2014); pectin derived oligosaccharides (POS); Babber et al (2015); Chung et al (2017); Strube et al (2015); and β-glucan derived oligosaccharides; Meyer et al (2015); Míguez et al (2016). Galacto-oligosaccharides (GOS) can be synthesized from lactose, and is also a new prebiotic oligosaccharide; Torres et al (2010). Chemical characteristics important for an oligosaccharide to have prebiotic effects or not, are the type of chemical bonds between sugar moieties, the identity of the sugar molecules, and their branching; Markowiak & Slizewska (2018); Pourabeden & Zhao (2015), Kim et al (2019).

The recommended amounts in an animal feed of MOS to swine is approx. 0.25 to 0.5% of the final feed formulation, and the recommended amounts of FOS is approx. 0.03 to 1.25% of the feed formulation. Maribo (2005): “Tilsætningsstoffer til svin. Landsudvalget for svin”. This supports the idea that even small amounts of prebiotic oligosaccharides added to a production animal feed make a difference.

In a second aspect, the invention relates to a method for the production of a feed or food ingredient according to the invention, comprising the following steps:
mixing soy hulls with one or more carbohydrase(s) selected from mannanase(s), pectinase(s), xylanase(s), glucanase(s), and cellulase(s);
hydrolysing the mixture under conditions, where the dry matter (DM) content is 55% by weight or less, in a time period of from 1 to 48 hours, at a temperature from 20 to 60° C.;
optionally fully inactivating the activity of the one or more carbohydrase(s); and
isolating the feed or food ingredient.

The invention further provides a feed or food product or a nutritional supplement for production animals containing from 0.5 to 99% by weight of the feed or food ingredient of the invention, such as a feed product for use in a diet for production animals, preferably newborn and young animals, such as piglets, calves, and poultry, in need of prebiotic oligosaccharides.

### Definitions

In the context of the current invention, the following terms are meant to comprise the following, unless defined elsewhere in the description.

The term “comprising” is to be interpreted as specifying the presence of the stated part(s), step(s), feature(s), composition(s), chemical(s), or component(s), but does not exclude the presence of one or more additional parts, steps, features, compositions, chemicals or components. E.g., a composition comprising a chemical compound may thus comprise additional chemical compounds, etc.

### Biomass:

Comprises biological material produced by the photosynthesis and that can be used in industrial production. In the present context, biomass refers to fibre-rich plant matter in the form of soy hulls. The soy hulls may be applicable in raw form or in a disintegrated and/or pretreated form, depending of its nature and of the skilled person's approach to a specific raw material.

### Soya Bean Products:

Refers to plant matter in the form of soya bean products, in the present context in particular products from soy hulls, and mixtures thereof. The soya bean can be from any soya bean source, such as from South or North America or Asia or Europe, and it can be of gene modified origin (GMO) or of non-gene modified origin (non-GMO).

### Soy Hulls

Soy hulls are a dedicated fibre source, high in total and in insoluble fibres and also contain some protein. Soy hulls are a by-product of soy bean processing and consist of the soy bean coating. Soy hulls contain complex carbohydrates, such as pectin, hemicelluloses, and cellulose, and is a good source of dietary fibres.

### Disintegrated:

Means, for example, disintegrated by chemical or physical means according to methods known in the art, e.g. milling, blending, cooking, and/or acid or alkaline treatment. The skilled person will know whether disintegration will be necessary and, if required, which disintegration may be appropriate for the specific plant biomass applied in the invention.

### Oligosaccharides:

An oligosaccharide is normally defined as a saccharide oligomer containing a small number (3-10) of component monomer sugars. In the context of the present invention, an oligosaccharide is defined broader, and it can be a saccharide oligomer containing from 3 to monomer sugar units.

### Oligosaccharides 3-30 Sugar Units/Oligosaccharides DP 3-30

In the context of the present invention, these terms designate saccharide oligomers containing 3-30 monomer sugar units and is a feature applied in the definition of the present new product. In the context of the present invention, soy oligosaccharides, in particular raffinose, stachyose, and verbascose that are included in the raw soy hulls, are not included in the herein defined DP 3-30.

### Prebiotic Oligosaccharides

An oligosaccharide to be regarded as prebiotic must not be hydrolysed or absorbed in the upper part of the gastrointestinal tract, and must be assimilated selectively by beneficial microorganisms in the colon, promoting beneficial luminal or systemic effects; Meyer et al (2015); Míguez et al (2016). They can be fermented by microorganisms in the hindgut, but need to “survive” until they reach there; Smiricky-Tjardes et al (2003).

### Monosaccharides/Mono Sugars

Monosaccharides, or mono sugars, are simple saccharide monomers, comprising five and/or six carbon atoms, and is the basic units of sugar and carbohydrates. In the context of the present invention, monosaccharides/mono sugars are in particular glucose, fructose, and galactose.

### Polysaccharides

Polysaccharides are saccharide polymers containing a large number of component monomer sugars, also known as complex carbohydrates. Polysaccharides may be soluble and insoluble.

### Dietary Fibres

Dietary fibres comprise soluble and insoluble, non-starch polysaccharides and may comprise oligosaccharides, lignin, and resistant starch. The raw biomasses selected in the context of the present invention only contain minor amounts of oligosaccharides and resistant starch, or are substantially free of resistant starch.

### Carbohydrase

Carbohydrase in the present context is any enzyme capable of hydrolysing any carbohydrate structure such as mannanase(s), pectinases(s), xylanase(s), glucanase(s), and cellulase(s).

### Yeast

In the present context, yeast may in particular be selected among Saccharomyces cerevisiae strains, including spent brewer's yeast and spent distiller's yeast and spent yeast from wine production, bioethanol yeast, or spent yeast from bioethanol production, baker's yeast, and yeast strains fermenting C5 sugars.

### Microorganisms

Microorganisms are organisms which are microscopic, making them too small to be seen by the unaided human eye. Microorganisms include bacteria, fungi, archaea, protists, and viruses.

### Lactic Acid Bacteria

Are usually found in decomposing plants and milk products and produce lactic acid as the major metabolic end product of carbohydrate bio-conversion. The lactic acid bacteria are genera of microorganism which produce organic acids, such as lactic acid and some acetic acid, as metabolic products of carbohydrate bio-conversion. The genera are in particular, but are not limited to, Lactobacillus, Pediococcus, Lactococcus, Enterococcus, Weisella, Streptococcus, and Leuconostoc.

### Other Genera

In the context of the present invention, other genera refer to the most relevant other bacterial genera in relation to the invention. They comprise a number of genera which also produce organic acids, such as lactic acid and acetic acid, as metabolic products of carbohydrate bio-conversion, but often to a lesser extent than the lactic acid bacteria. In the context of the present invention other genera than the lactic acid comprises, but are not limited to, Bacillus, Bifidobacterium, Brevibacillus, Propionibacterium, Clostridium, and Geobacillus. Certain strains are used as probiotics.

### Feed Products

Comprise ready-to-use feed or feed ingredients for production animals such as piglets, calves, poultry, furred animals, and sheep.

### Food Products

Comprise ready-to-use food or food ingredients for human nutrition.

The invention is illustrated in the drawing, wherein:

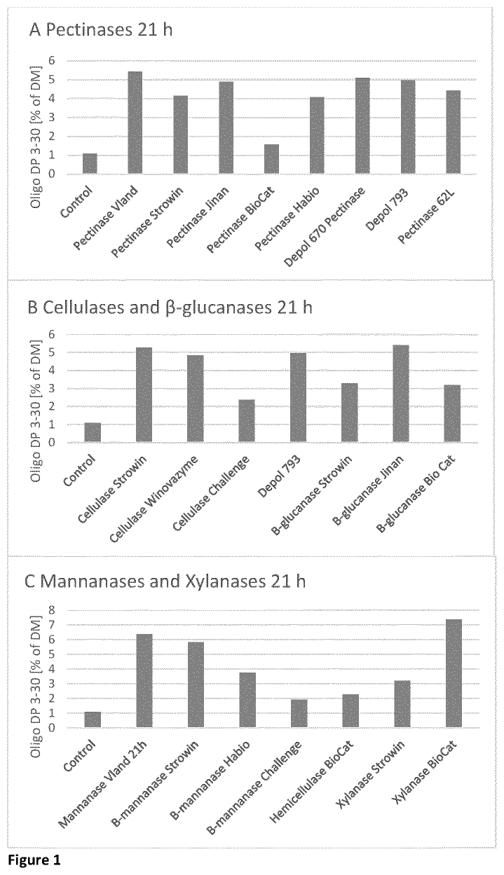

FIG. 1 shows a first screening results after incubation of soy hull biomass with different carbohydrate targeting enzymes under pH regulation. Oligosaccharides DP 3-30 measured do not include raffinose, stachyose, and verbascose.

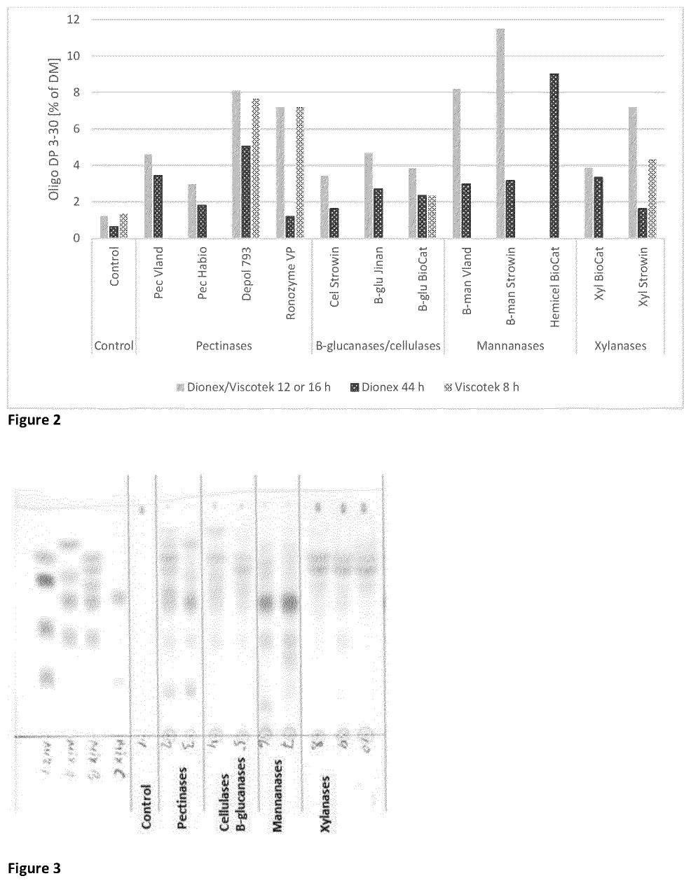

FIG. 2 shows a similar screening after a similar incubation of soy hull biomass without pH regulation, but with baker's yeast added. Oligosaccharides DP 3-30 measured do not include raffinose, stachyose, and verbascose.

FIG. 3 shows TLC results of incubation of soy hulls with different carbohydrase enzymes.

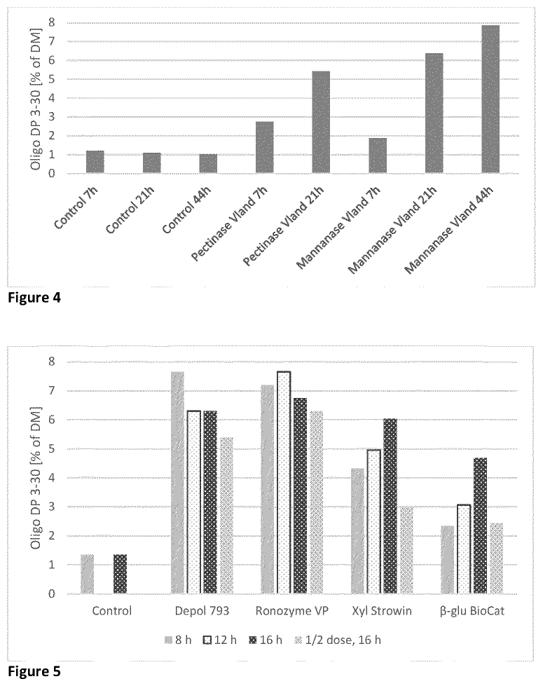

FIG. 4 shows the effect of hydrolysis time when Vland pectinase or mannanase is used for incubation of the soy hull biomass. Oligosaccharides DP 3-30 measured do not include raffinose, stachyose, and verbascose.

FIG. 5 shows the effect of hydrolysis time and enzyme dose when soy hull biomass is incubated with different carbohydrase enzymes. Oligosaccharides DP 3-30 measured do not include raffinose, stachyose, and verbascose.

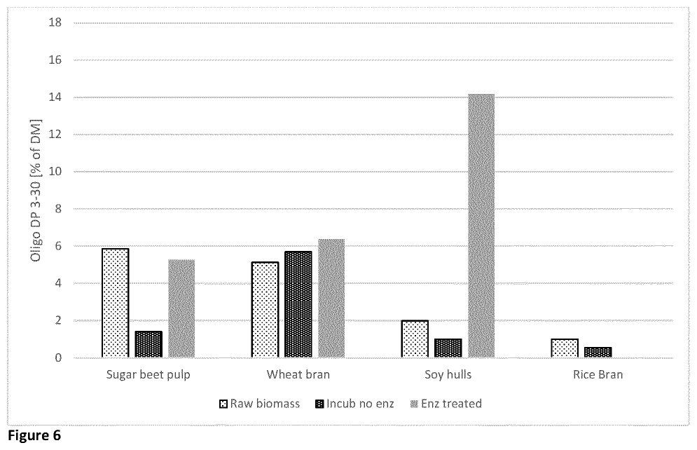

FIG. 6 shows as a comparison quantification of oligosaccharides DP 3-30 (not include raffinose, stachyose, and verbascose), in different biomasses, either the raw biomass, an incubated biomass without enzymes, or enzyme treated biomasses after incubation with carbohydrase enzyme.

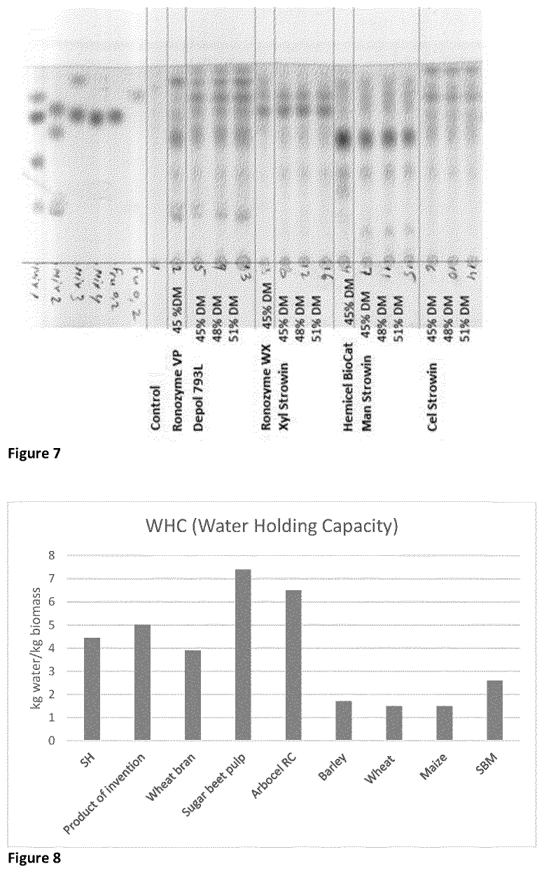

FIG. 7 shows TLC for a soy hulls biomass after incubation with Depol 793L (pectinase), xylanase, mannanase, and cellulase from Strowin, respectively.

FIG. 8 shows water holding in soy hulls in comparison with different biomasses, either in raw form or after treatment according to the invention.

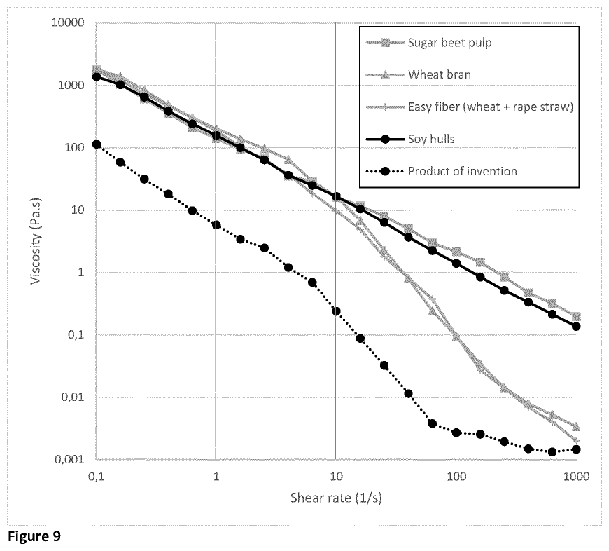

FIG. 9 shows viscosity in different biomasses, either in raw form or after treatment according to the invention.

### DETAILED DESCRIPTION OF THE INVENTION

### The Product of the Invention in its First Aspect:

In its first aspect, the invention relates to a feed or food ingredient derived from soy hulls, wherein the feed or food ingredient comprises dietary fibres from the soy hulls in the form of soluble and insoluble polysaccharides, and wherein the dietary fibres from the soy hulls have been partly degraded by one or more carbohydrase(s) selected from mannanase(s), pectinase(s), xylanase(s), glucanase(s), and cellulase(s), into oligosaccharides having from 3 to 30 monomer sugar units (Oligosaccharides DP 3-30), and wherein the feed or food product comprises 5% by weight monosaccharides or less.

In this context, the oligosaccharides DP 3-30 derived from the dietary fibres do not comprise raffinose, stachyose and verbascose.

In one embodiment of this aspect, the dietary fibres from soy hulls have been partly degraded by the one or more carbohydrase(s) into oligosaccharides providing prebiotic effect.

In any embodiment, the feed or food product comprises 5% by weight or less monosaccharides; such as 4% by weight or less; 3% by weight or less; 2% by weight or less; 1% by weight or less; 0.5% by weight or less; is substantially free of monosaccharides, or is free of monosaccharides.

In another embodiment, the feed or food ingredient may comprise 4% by weight or more of the oligosaccharides having from 3 to 30 monomer sugar units (Oligosaccharides DP 3-30), such as 5% by weight or more, 6% by weight or more, 7% by weight or more, 8% by weight or more, 9% by weight or more, 10% by weight or more, 12% by weight or more, 15% by weight or more, or 20% by weight or more, of oligosaccharides having from 3 to 30 monomer sugar units (DP3-30). In this context, the oligosaccharides DP 3-30 derived from the dietary fibres do not comprise raffinose, stachyose, and verbascose.

In any of the embodiment of the invention in its first aspect, the feed or food ingredient may sometimes further comprise live yeast, such as live yeast selected among Saccharomyces cerevisiae strains, including spent brewer's yeast, baker's yeast, spent distiller's yeast, and spent yeast from wine production, bioethanol yeast, or spent yeast from bioethanol production, and yeast strains fermenting C5 sugars. The live yeast may e.g. be present in an amount of from 0.05 to 10%, such as 0.10%, 0.15%, 0.20%, 0.25%, 0.5%, 1%, 1.5%, 2%, 2.5%, 3%, 3.5%, 4%, 4.5, 5%, 6%, 7%, 8% or 9%.

In any of the embodiment of the invention in its first aspect, the oligosaccharides raffinose, stachyose, and/or verbascose present in the raw soy hulls may sometimes have been fully or partly degraded.

In any of the embodiment of the invention in its first aspect, the feed or food ingredient may sometimes further comprise one or more microorganisms and/or metabolic products of carbohydrate degradation of the soy hulls by one or more microorganisms.

In any of the embodiment of the invention in its first aspect, the feed or food ingredient may sometimes further comprise one or more microorganisms selected from lactic acid bacteria and/or metabolic products of carbohydrate degradation of the soy hulls by lactic acid bacteria, such as Lactobacillus, Pediococcus, Lactococcus, Enterococcus, Weisella, Streptococcus, and Leuconostoc.

In any of the embodiment of the invention in its first aspect, the feed or food ingredient may sometimes further comprise one or more microorganisms and/or metabolic products of carbohydrate degradation of the soy hulls by the one or more microorganisms selected from Bacillus, Bifidobacterium, Brevibacillus, Propionibacterium, Clostridium, and Geobacillus.

In any of the embodiment of the invention in its first aspect, the feed or food ingredient may be derived from soy hulls, wherein the soy hull fibres have been degraded by one or more β-mannanase(s) into oligosaccharides, comprising 4% by weight or more of oligosaccharides having from 3 to 30 monomer sugar units, such as 5% by weight or more, 10% by weight or more, 15% by weight or more, or 20% by weight or more, of oligosaccharides having from 3 to 30 monomer sugar units (DP3-30). In this context, the oligosaccharides DP 3-30 derived from the dietary fibres do not comprise raffinose, stachyose, and verbascose.

In any of the embodiment of the invention in its first aspect, the feed or food ingredient may be derived from soy hulls, wherein the soy hull fibres have been degraded by one or more pectinase(s) into oligosaccharides, comprising 4% by weight or more of oligosaccharides having from 3 to 30 monomer sugar units, such as 5% by weight or more, 10% by weight or more, 15% by weight or more, or 20% by weight or more, of oligosaccharides having from 3 to 30 monomer sugar units (DP3-30). In this context, the oligosaccharides DP 3-30 derived from the dietary fibres do not comprise raffinose, stachyose, and verbascose.

In any of the embodiment of the invention in its first aspect, the feed or food ingredient may be derived from soy hulls, wherein the soy hull fibres have been degraded by one or more xylanase(s) into oligosaccharides, comprising 4% by weight or more of oligosaccharides having from 3 to 30 monomer sugar units, such as 5% by weight or more, or 10% by weight or more, of oligosaccharides having from 3 to 30 monomer sugar units (DP3-30). In this context, the oligosaccharides DP 3-30 derived from the dietary fibres do not comprise raffinose, stachyose, and verbascose.

In any of the embodiment of the invention in its first aspect, the feed or food ingredient may be derived from soy hulls, wherein the soy hull fibres have been degraded by one or more glucanase(s) into oligosaccharides, comprising 4% by weight or more of oligosaccharides having from 3 to 30 monomer sugar units, such as 5% by weight or more, or 10% by weight or more, of oligosaccharides having from 3 to 30 monomer sugar units (DP3-30). In this context, the oligosaccharides DP 3-30 derived from the dietary fibres do not comprise raffinose, stachyose, and verbascose.

In any of the embodiment of the invention in its first aspect, the feed or food ingredient may be derived from soy hulls, wherein the soy hull fibres have been degraded by several of the carbohydrases into oligosaccharides having from 3 to 30 monomer sugar units (DP3-30).

### The Method of the Invention in its Second Aspect:

In the second aspect of the invention, it relates to a method for the production of feed or food ingredient according to the invention, comprising the following steps:
mixing soy hulls with one or more carbohydrase(s) selected from mannanase(s), pectinase(s), xylanase(s), glucanase(s), and cellulase(s);
hydrolysing the mixture under conditions, where the dry matter (DM) content is 55% by weight or less, in a time period of from 1 to 48 hours, at a temperature from 20 to 60° C.;
optionally fully inactivating the activity of the one or more carbohydrase(s);
isolation of the feed or food ingredient.

The skilled person will be able to select a suitable amount of carbohydrase enzyme(s) that fulfils the requirement of the method, depending on the process conditions, the intended result, and the optimal process economy.

In one embodiment of this aspect, the fibre-rich plant biomass may have been disintegrated before being mixed with the one or more carbohydrase(s). The means for disintegration may be physical or chemical, and the skilled person will know whether disintegration is adequate or necessary, and which means are applicable for a specific biomass.

In any embodiment of the inventions in its second aspect, the method includes a step of full inactivating the activity of the one or more carbohydrase(s), conducted after the step of hydrolysing the mixture. The inactivation step secures that the Oligosaccharides DP 3-30 are not degraded into monosaccharides.

In any embodiment of the invention in its second aspect, the dry matter content is 55% by weight or less, such as 53% by weight or less, 51% by weight or less, 50% by weight or less, 48% by weight or less, 46% by weight or less, or 45% by weight or less.

In any embodiment of the invention in its second aspect, the reaction time is from 1 to 48 hours, such as 2, 4, 6, 8, 12, 16, 20, 24, 28, 32, 36, 40 or 44 hours.

In any embodiment of the invention in its second aspect, the reaction temperature is from 20 to 60° C., such as 25, 30, 32, 35, 37, 40, 45, 50, 55, or 58° C.

In any embodiment of the invention in its second aspect, the skilled person will be able to select and adapt reaction time and reaction temperature in proportion to the selected carbohydrase(s) and the amount thereof.

In any embodiment of the invention in its second aspect, yeast, such as live yeast selected among Saccharomyces cerevisiae strains, including spent brewer's yeast, baker's yeast, spent distiller's yeast, and spent yeast from wine production, bioethanol yeast, or spent yeast from bioethanol production, and yeast strains fermenting C5 sugars, may be added to the mixture of soy hulls and carbohydrase(s) before the hydrolysing step, e.g. in an amount of from 0.05 to 10%, such as 0.1%, 0.15%, 0.20%, 0.25%, 0.5%, 1%, 1.5%, 2%, 2.5%, 3%, 3.5%, 4%, 4.5, 5%, 6%, 7%, 8% or 9%. The skilled person will be able to select a suitable amount, depending on the process conditions and the intended result.

In any embodiment of the invention in its second aspect, one or more microorganism(s) may be added to the mixture of soy hulls and carbohydrase(s) before the hydrolysing step. Addition of microorganism(s) is suitable for conversion of carbohydrate present in the soy hulls into useful metabolic products, in particular organic acids, such as lactic acid and acetic acid. The skilled person will be able to select a suitable amount, depending on the process conditions and the intended result.

In any embodiment of the invention in its second aspect, lactic acid bacteria, such as Lactobacillus, Pediococcus, Lactococcus, Enterococcus, Weisella, Streptococcus, and Leuconostoc may be added to the mixture of soy hulls and carbohydrase(s) before the hydrolysing step. Addition of lactic acid bacteria is suitable for conversion of carbohydrate present in the plant biomass into useful metabolic products, in particular organic acids, such as lactic acid and acetic acid. The skilled person will be able to select a suitable amount, depending on the process conditions and the intended result.

In any embodiment of the invention in its second aspect, one or more microorganism(s), selected from Bacillus, Bifidobacterium, Brevibacillus, Propionibacterium, Clostridium, and Geobacillus, may be added to the mixture of soy hulls and carbohydrase(s) before the hydrolysing step. The skilled person will be able to select a suitable amount, depending on the process conditions and the intended result.

In any embodiment of the invention in its second aspect, α-galactosidase may be added to the mixture of soy hulls and carbohydrase(s) before the hydrolysing step. Addition of α-galactosidase is suitable for degrading raffinose, stachyose, and verbascose present in soy hulls. The skilled person will be able to select a suitable amount, depending on the process conditions, and the intended result.

### The Feed or Food Product or Nutritional Supplement of the Invention in its Third Aspect:

In a third aspect of the invention, it relates to feed or food product or a nutritional supplement containing from 0.5 to 99% by weight of a feed or food ingredient according to the invention.

In any embodiment of the invention in its third aspect, the feed product or nutritional supplement may be for use in a diet for production animals, such as a diet for improving performance in production animals, in particular newborn and young animals, such as piglets, calves, and poultry.

In any embodiment of the invention in its third aspect, the feed product or nutritional supplement may be for use in a diet for production animals, in particular newborn and young animals, such as piglets, calves, and chickens, in need of prebiotic oligosaccharides.

In a 4th aspect of the invention, it relates to use of a feed ingredient according to the invention in a diet for production animals, in particular newborn and young animals, such as piglets, calves, and chickens.

### EXAMPLES

### Material and Methods

### Enzymes and Enzyme Suppliers:

BIO-CAT, Vland Biotech Inc, Strowin, Jinan BestZyme, Winovazyme, Habio, Challenge Group, Biocatalysts Ltd and DSM. Enzymes were supplied between April 2018 to December 2019 and the specific names of the enzymes are seen below.

| | Pectinase | Xylanase | Mannanase | β-glucanase | Cellulase | Blend |
|---|---|---|---|---|---|---|
| BioCat | Pectinase | Xylanase | | β-glucanase | Cellulase AN | Hemi- |
| | endo 3.5K | 150K | | 6K | 100K | cellulase |
| | | | | | Cellulase | 400K |
| | | | | | 150K | |
| Vland | Pectinase | Xylanase | Neutral | β-glucanase | Neutral- | |
| | 300K | 200K | mannanase | 50K | cellulase | |
| | | | 3K | | 30K | |
| Strowin | Pectinase | Xylanase | β-mannanase | β-glucanase | Cellulase | |
| | 30K | 200K | (normal) 50K | 50K | 10K | |
| Jinan | Pectinase | Xylanase | β-mannanase | β-glucanase | Cellulase | |
| | 30K | 600K | 100K | 100K | 20K | |
| Winovazyme | | Xylanase | β-mannanase | β-glucanase | Cellulase | |
| | | 200K | CM 100K | 50K | 8K | |
| DSM | Ronozyme | Ronozyme | | | | |
| | VP | WX | | | | |
| Habio | Pectinase | | β-mannanase | β-glucanase | Cellulase | |
| | 30K | | 100K | 50K | 20K | |
| Challenge | | Xylanase | β-mannanase | β-glucanase | Cellulase | |
| | | 50K | 50K | 50K | 20K | |
| Biocatalysts | Pectinase | Depol | | | Cellulase | Depol 793L |
| | 62L | 761P | | | 13L | Depol 670L |

### Thin Layer Chromatography (TLC Method)

A 10% DM suspension of the sample was first prepared and homogenized for 30 sec using a Ultra Thorax instrument at level 2. The sample was stirred at room temperature for 30 min, and centrifugation at 3000 G for 10 min. The supernatant was used directly for TLC.

1.4 μl of samples, and different standard mixes, were added to the TLC plate. After drying, the plate was placed in the chromatography tank for 50-60 min, followed by drying and dipping in developer liquid for 2 second. After drying, TLC patterns became visible after a short time at 150° C. Chromatography fluid consisted of demineralized water with 46% vol/vol n-butanol and 31% vol/vol pyridine. Developer solution consisted of 2 g diphenylamine, 84 ml acetone, 2 ml aniline and 15 ml 85% phosphoric acid per 100 ml.

### Ion-Exchange Chromatographic Analysis (Dionex)

A 10% DM suspension of the sample was prepared and homogenized for 30 sec using an Ultra Turrax T25 instrument, IKA, at level 2. The sample was stirred at room temperature for 30 min, and centrifugation at 3000 G for 10 min. The supernatant was used for Dionex or Viscotek analysis.

Oligosaccharides and sugars were separated and quantified by high-performance anion exchange chromatography with pulsed amperometric detection (HPAEC-PAD, Dionex). Oligosaccharides were estimated using an external maltodextrin standard. Samples were injected on a CarboPacPA-200 column using 0.4 mL min-1 flow rate, 150 mM isocratic NaOH and the following NaOAc gradient profile: 0-5 min: 0-110 mM linear gradient, 5-30 min: 110-350 mM convex gradient.

### Size Exclusion Chromatographic Analysis (Viscotek)

Samples were examined by size exclusion chromatography (SEC) using a Viscotek System (Malvern, UK) equipped with two columns in series: GS-320 HQ and GS-620 HQ columns (Shodex) attached to a TDA 302 module (Triple detector array) comprising a refractive index detector (RI), a four-bridge viscometer detector (VIS) and a light scattering detector (LS). The LS comprised a right-angle light scattering (RALS) and a low angle light scattering (LALS) that measures the scattered light at 7° and 90° with respect to the incident beam. The instrument was calibrated using pullulan (50 kDa, polydispersity 1.07, Showa Denko) solubilized in MilliQ water (1 mg/ml) at 99° C. for 120 min at 1000 rpm. Elution was performed with 50 mM ammonium formate (HCO₂NH₄) buffer and 0.5 mL min-1 flow rate. Samples were filtered through a 0.22 μm centrifuge filter and 50 μl sample injected (GPCmax module) into the column and separated at 60° C. Authentic well-characterised linear maltooligosaccharides were used as external standards. Data were analysed using the OmniSec Software 4.7 (Malvern Instrument, Ltd.).

### Example 1

### Small Scale Screening Showing the Effect of Carbohydrases in Soy Hulls

A total of 40 commercial carbohydrase enzymes covering at least the general groups of mannanases, xylanases, cellulases, β-glucanases, and pectinases, from different suppliers were tested in soy hull biomass. The enzymes were dosed according to enzyme price, adding a dose corresponding to a fixed price per weight of soy hull dry matter, and compared accordingly. Soy hulls were delivered as dry pills, and were roughly disintegrated in a blender. Enzymes and soy hulls were mixed and incubated at a dry matter (DM) content of 45-48% by weight, at 32° C. for 8-44 hours. Approx. 90 g soy hulls were used in this small scale screening. α-galactosidase was added to all samples to remove the oligosaccharides stachyose and raffinose in the soy hull biomass. In some experiments, baker's yeast was added to the reaction mixture. In some experiments, pH was regulated to 4.5 with H₂SO₄.

After hydrolysis of the biomass, its content of Oligosaccharide DP 3-30 (oligosaccharides having 3-30 sugar unit) was determined with either the Dionex or the Viscotek method. The TLC method was also applied for further quantification of the pattern of enzyme-cutting into oligosaccharides by the different enzymes.

FIGS. 1A, B and C show the first screening results after incubation for 21 hours with pH-regulation and analysis of the product with the Dionex method.

FIG. 2 shows the screening results after incubations for 8-44 hours without pH-regulation and analysis of the product with the Dionex and Viscotek method. If more analysis was done with the same enzyme, means were used in the figure.

FIG. 3 shows sugar compositions of soy hulls subjected to treatment with different carbohydrase enzymes, clearly showing that different types of enzymes cut differently in the soy hulls, resulting in typical patterns for mannanase, xylanase, pectinase, and β-glucanase/cellulose, respectively. The reference sugar mixtures displayed in lane 1-4 contain as follows reading from the top:
Mix 1: xylose, sucrose, raffinose, stachyose
Mix A: arabinobiose, arabinose, mannobiose, mannotriose
Mix B: xylose, xylobiose, xylotriose, mannobiose, mannotriose
Mix C: galactose, galacturonic acid

The mean maximal obtained oligosaccharide levels obtained in the small scale screening with the different enzyme groups after 16 hours of hydrolysis, are shown as a mean analysis value for the best enzyme below:

| | Oligosaccharides |
|---|---|
| | DP 3-30, % by |
| | weight of DM |
| Control | 1 |
| B-mannanase | 11 |
| Pectinase | 9 |
| Xylanase | 8 |
| Glucanase/cellulase | 5 |

### Example 2

### Showing the Effect of Carbohydrases in Soy Hulls in Pilot Scale

Three hydrolysis experiments was conducted in a horizontal, slowly turning reactor, applying 450 kg soy hull per pilot experiment and the most effectively working enzymes from the screening example, and baker's yeast and α-galactosidase. Soy hull pills were not disintegrated beforehand, the initial DM and temperature were 48% by weight and 30° C., and the incubation time was 16 hours.

Viscotek analysis was performed. The content of Oligosaccharide DP 3-30 (oligosaccharides having 3-30 sugar unit) was recalculated to % by weight of the total soy hull biomass.

| | Oligosaccharides |
|---|---|
| | DP 3-30, % by |
| | weight of DM |
| Best pectinase enzyme | 15.8 |
| Best mannanase enzyme | 22.8 |
| Best xylanase enzyme | 17.0 |

### Example 3

### Showing the Effect of Incubation Time and Enzyme Dose

The effect of incubation time was examined using either a hydrolysis condition with pH at 4.5 (FIG. 4) or an unregulated pH (FIG. 5), as described in example 1. FIG. 5 also shows the effect of including half enzyme dose. As would be expected, the experiments show that the effect of incubation time is enzyme dependent.

### Example 4

Showing Oligosaccharide Levels and the Effect of Carbohydrases in Other Biomasses in Comparison with Soy Hulls

The level of oligosaccharides was tested in a range of different biomasses, in different forms:
a) the raw biomass (control);
b) an incubated biomass without added carbohydrase enzymes (α-galactosidase was added to biomasses containing raffinose, stachyose, and verbascose, in order to remove these oligosaccharides); or
c) the biomass incubated with commercial carbohydrase enzymes.

The enzymes were dosed according to price, adding a dose corresponding to a fixed price per weight of biomass dry matter, and compared accordingly. The biomasses sugar beet pulp and soy hulls were delivered as dry pills and were roughly disintegrated in a blender. The other biomasses were used as they were delivered. Enzymes, baker's yeast, and biomass were mixed and incubated at 45 or 48% DM at 32 or 37° C. for 16, 20 or 44 hours. After hydrolysis, the biomass was analysed in the same way as explained in example 1. Results are seen in FIG. 6. Soy oligosaccharides (raffinose, stachyose and verbascose), if present in the raw biomass, have been subtracted.

FIG. 6 shows Dionex or Viscotek quantification of oligosaccharides in the different biomasses, either the raw biomass, an incubated biomass without enzymes, or enzyme treated biomass.

Comparing the release of oligosaccharides on a relative basis (compared to start level in raw material), soy hulls stands out as a biomass where it is possible to release the highest relative amount.

| | Increase factor in |
|---|---|
| | oligosaccharides |
| | DP 3-30 compared |
| | to raw material |
| | (exclusive soy |
| | oligosaccharides) |
| Sugar beet pulp | 0.9 |
| Wheat bran | 1.2 |
| Soy hulls | 7.2 |

### Example 5

### Showing Effect of Incubation DM in Soy Hulls

An experiment performed as described in example 1 with non-regulated pH, was performed to test three starting biomass DM levels: 45, 48 and 51% of DM. The results are seen in FIG. 7, showing a TLC, indicating no effect of increasing DM % from 45% to 51% with Depol 793L (pectinase), xylanase, mannanase and cellulase from Strowin.

### Example 6

A piglet feeding trail was conducted May-August 2018 at a Danish test station: “Skjoldborg teststation, TestGris, SvineRådgivningen, Herning, Denmark”. Four diets were tested, using a conventional pig production system and comprising the following:
1. A control with no soy hulls;
2. FaserGold, an extruded soy hull, amount of 2.5% by DM weight;
3. A soy hull incubated with α-galactosidase and baker's yeast for 8 hours, milled and dried, amount of 2% by DM weight;
4. A soy hull incubated with α-galactosidase, Ronozyme VP, and baker's yeast for 7 hours, milled and dried, amount of 2% by DM weight.

The trial ran for 6 weeks following weaning, three separate phases (A, B, and C) of 2 weeks each. The soy hull products were only included during phase A. In phase B and C, piglets were fed the same feed. None of the diets contained antibiotics or therapeutic levels of veterinary ZnO. All diets were fed ad libitum. The pigs had permanent access to fresh water. The diets in phase A were wheat (35-38%) and barley (15%) based, using HP300 from Hamlet Protein (19%) as protein source. An equal amount of “premix” was used in each diet (25%), and soy oil was used to equalize energy level in the diets (2.7-3.7%). Wheat and barley were milled, and the diet pelletized. Phase B and C diets were produced by TestGris, following Danish feeding standards.

4111 Danbred crossbred piglets were used, 25±3 days of weaning age, and an average body weight of 6.4 kg. 64 double pens were used. Approx. 2*32 piglets were used in each double pen. The double pens were allocated to one of the 4 diets.

Measurement of the output was done as average daily gain (ADG), feed intake (FI), and feed utilization (FU) (also known as feed conversion rate (FCR). The results as shown in Table 1.

TABLE 1
Average daily gain (ADG, g), feed intake (FI) and feed
utilisation (FU) in phase A (6-9 kg) phase B (9-15
kg), phase C (15-30 Kg) and the whole test period (A-C)
of pigs fed the four experimental diets.^{x}

| | Phase | Diet 1 | Diet 2 | Diet 3 | Diet 4 | P-value |
|---|---|---|---|---|---|---|
| ADG, g/d | A | 196 | 200 | 198 | 210 | 0.08 |
| | B | 533 | 531 | 540 | 526 | 0.71 |
| | C | 794 | 792 | 807 | 785 | 0.70 |
| | A-C | 497 | 495 | 507 | 499 | 0.47 |
| FI, g/d | A | 241 | 242 | 239 | 246 | 0.66 |
| | B | 692 | 695 | 695 | 691 | 0.98 |
| | C | 1227 | 1187 | 1224 | 1220 | 0.17 |
| | A-C | 710 | 696 | 709 | 709 | 0.45 |
| FU, | A | 1.25^{b} | 1.23^{ab} | 1.20^{ab} | 1.18^{a} | 0.005 |
| kg feed/ | B | 1.31 | 1.30 | 1.29 | 1.32 | 0.14 |
| kg gain | C | 1.56 | 1.55 | 1.52 | 1.57 | 0.62 |
| | A-C | 1.42 | 1.41 | 1.40 | 1.42 | 0.43 |

^{x}Values are LS-means (n = 16).
^{ab}LS-Means within rows without a common superscript differ (P < 0.05).

In general, piglets maintained good health during the experiment.

It is concluded that ADG tended to be significantly affected by the dietary treatments in phase A with the highest ADG in the Diet 4 group. The FI was not affected by the dietary treatments, but the FU was significantly higher in the Diet 4 group compared with the Diet 1 group. The different diets fed in phase A did not affect the performance in phase B or C.

### Example 7

Parameters as water holding capacity (WHC) as well as viscosity are important in fibre products. A relatively high water holding capacity in a fibre product ensures satiety feeling in the animal and helps ensure an optimal stool consistency. Viscosity is often an aspect of fibres that give rise to concern, especially in poultry feeding, where a high viscosity is unwanted, as it can slow nutrient absorption and give better room for pathogens to proliferate in the gut. Water holding capacity is measured as the amount of water which can be hold by the biomass against gravity, without release of free water, and the unit is kg water/kg biomass.

### Water Holding Capacity (WHC)

WHC was measured in a range of dry fibre products, including raw soy hulls, and a finished fibre ingredient according to the present invention, viz. a pectinase treated soy hull as well as barley, wheat, maize, and soy bean meal (SBM). All samples were first disintegrated through a 500 μm sieve. Results are seen in FIG. 8, and show that the product of the invention have a nicely high WHC, as also characterize many other fibre products.

### Viscosity

For the viscosity measure the following method was applied: All samples was milled through a 500 μm sieve. 1 g sample and 20 mL 0.05M phosphate buffer (pH 7) were incubated at 40 C.° for 0.5 h. After that, samples were centrifuged at 500 rpm (31 G) for 10 min, in a Allegar X-22R centrifuge, Beckman Coulter, before the rheology analysis. The samples were analysed on a Discovery HR-3 Rheometer (TA Instruments). A finished fibre ingredient according to the present invention, viz. a pectinase treated soy hull is compared to raw soy hulls, sugar beet pulp, wheat bran and Easy Fibre (a blend of rape straw and wheat straw). Results are seen in FIG. 9 and show that compared to raw soy hulls, wheat bran, sugar beet pulp and Easy Fibre, the product of the invention has reduced viscosity. Shear rates relevant in the gut are approximately between 1 and 10 s-1 is indicated in the figure.

### REFERENCES

Aachary & Prapulla (2011). Xylooligosaccharides (XOS) as an Emerging Prebiotic: Microbial Synthesis, Utilization, Structural Characterization, Bioactive Properties, and Applications. Comprehensive Reviews in Food Science and Food Safety Vol. 10, 2011
Babber et al 2015. Pectic oligosaccharides from agricultural by-products: production, characterization and health benefits. Crit Rev Biotechnol: 1-13
Dotsenko et al (2017). Enzymatic production of wheat and ryegrass derived xylooligosaccharides and evaluation of their in vitro effect on pig gut microbiota. Biomass Conversion and Biorefinery
Chung et al (2017). Prebiotic potential of pectin and pectic oligosaccharides to promote anti-inflammatory commensal bacteria in the human colon. FEMS Microbiology Ecology, 93
Corrigan et al (2015). Phylogenetic and Functional Alterations in Bacterial Community Compositions in Broiler Ceca as a Result of Mannan Oligosaccharide Supplementation. Applied and Environmental Microbiology May 2015 Volume 81 Number 10
Englyst H N, Quigley M E and Hudson G J (1994) Analyst, 119, 1497-1509
Gaggia et al (2010). Probiotics and prebiotics in animal feeding for safe food production. International Journal of Food Microbiology 141 pp 15-28
Ghoddusi et al (2007). In vitro study on gas generation and prebiotic effects of some carbohydrates and their mixtures. Anaerobe 13 pp 193-199
Kiarie et al (2013). The role of added feed enzymes in promoting gut health in swine and poultry. Nutrition Research Reviews 26, 71-88
Kim et al (2011). Effect of dietary prebiotic supplementation on the performance, intestinal microflora, and immune response of broilers. Poultry Science 90:75-82
Kim et al (2019). Potential for Prebiotics as Feed Additives to Limit Foodborne Campylobacter Establishment in the Poultry Gastrointestinal Tract. Frontiers in Microbiology. Volume 10, Article 91
Kurakake et al (2006). Production of Galacto-manno-oligosaccharides from Guar Gum by β-Mannanase from Penicillium oxalicum SO. J. Agric. Food Chem. 54, 7885-7889
Liu et al (2018). Effect of probiotics and xylo-oligosaccharide supplementation on nutrient digestibility, intestinal health and noxious gas emission in weanling pigs. Asian-Australas J Anim Sci Vol. 31, No. 10 pp 1660-1669
Markowiak & Slizewska (2018). The role of probiotics, prebiotics and synbiotics in animal nutrition. Gut Pathog 10:21
Maribo (2005). Tilsætningsstoffer til svin. Landsudvalget for svin.
Meyer et al (2015). Biotechnological production of oligosaccharides-application in the food industry. Chapter 2 in: Food production and industry www.intechopen.com/books/food-production-and-industry
Míguez et al (2016). Pectic oligosaccharides and other emerging prebiotics. Chapter 15 in: Probiotics and prebiotics in human nutrition and health www.intechopen.com/books/probiotics-and-prebiotics-in-human-nutrition-and-health
Molist et al (2009) Effects of the insoluble and soluble dietary fibre on the physicochemical properties of digesta and the microbial activity in early weaned piglets. Animal Feed Science and Technology 149 pp 346-353
Moura et al (2018). In vitro fermentation of selected xylo-oligosaccharides by piglet intestinal microbiota. Food Science and Technology 41 pp 1952-1961
Nielsen et al (2014). Diets high in resistant starch and arabinoxylan modulate digestion processes and SCFA pool size in the large intestine and faecal microbial composition in pigs. British Journal of Nutrition 112, 1837-1849
Roberfroid et al (2010). Prebiotic effect: metabolic and health effects www.cambridge.org/core/services/aop-cambridge-core/content/view/F644C98393E2B3EB64A562854115D368/S0007114510003363a.pdf/pre biotic_effects_metabolic_and_health_benefits.pdf
Pourabeden & Zhao (2015). MINIREVIEW—Environmental Microbiology. Prebiotics and gut microbiota in chickens FEMS Microbiology Letters, 362
Probert el at (2004). Polydextrose, Lactitol, and Fructo-Oligosaccharide Fermentation by Colonic Bacteria in a Three-Stage Continuous Culture System. Applied and Environmental Microbiology vol 70 no 8 pp 4505-45
Scapini et al (2018). Nutritional Evaluation of Soybean Hulls with or without β-Mannanase Supplement on Performance, Intestinal Morphometric and Carcass Yield of Broilers Chickens. Brazilian Journal of Poultry Science
Smiricky-Tjardes et al (2003). In vitro fermentation characteristics of selected oligosaccharides by swine fecal microflora. J. Anim. Sci. 2003. 81:2505-2514
Strube et al (2015). In Situ Prebiotics for Weaning Piglets: In Vitro Production and Fermentation of Potato Galacto-Rhamnogalacturonan. Applied and Environmental Microbiology vol 81 no 5 pp 1668-78
Torres et al (2010). Galacto-oligosaccharides. Production, properties, applications and significance as prebiotics. Comprehensive reviews in food science and food safety. onlinelibrary.wiley.com/doi/pdf/10.1111/j.1541-4337.2010.00119.x
Zivkovic et al 2011. PREBIOTICS IN NUTRITION OF SOWS AND PIGLETS. Biotechnology in Animal Husbandry 27 (3), p 547-559

1-18. (canceled)

19. A feed or food ingredient derived from soy hulls,
wherein the feed or food ingredient comprises dietary fibres from soy hulls in the form of soluble and insoluble polysaccharides, wherein part of the dietary fibres from the soy hulls have been degraded by one or more carbohydrase(s) selected from mannanase(s), pectinase(s), xylanase(s), glucanase(s), and cellulase(s) into oligosaccharides having from 3 to 30 monomer sugar units (Oligosaccharides DP 3-30), wherein the feed or food ingredient comprises 4% by weight or more of said Oligosaccharides DP 3-30, as calculated without including raffinose, stachyose, and verbascose,
wherein the feed or food ingredient further comprises yeast,
with the proviso that the feed or food ingredient does not comprise more than 5% by weight monosaccharides.

20. The feed or food ingredient according to claim 19, wherein part of the dietary fibres from soy hulls have been degraded by the one or more carbohydrase(s) into Oligosaccharides DP 3-30 providing a prebiotic effect.

21. The feed or food ingredient according to claim 19, comprising 5% by weight or more of said Oligosaccharides DP 3-30.

22. The feed or food ingredient according to claim 19, wherein the yeast comprises one or more Saccharomyces cerevisiae strains.

23. The feed or food ingredient according to claim 19, wherein the yeast comprises one or more yeasts selected from spent brewer's yeast, spent distiller's yeast, spent yeast from wine production, bioethanol yeast, spent yeast from bioethanol production, baker's yeast, and yeast strains fermenting C5 sugars.

24. The feed or food ingredient according to claim 19, wherein the yeast is present at an amount of from 0.05 to 10%.

25. The feed or food ingredient according to claim 19, wherein raffinose, stachyose, and verbascose present in the soy hulls have been fully or partly degraded.

26. The feed or food ingredient according to claim 19, further comprising one or more selected from (i) one or more microorganisms and (ii) metabolic products of carbohydrate degradation of the soy hulls by the one or more microorganisms.

27. The feed or food ingredient according to claim 26, wherein the one or more microorganisms comprise lactic acid bacteria.

28. A method for producing the feed or food ingredient according to claim 19, comprising:
mixing soy hulls with one or more carbohydrase(s) selected from mannanase(s), pectinase(s), xylanase(s), glucanase(s), and cellulase(s) to obtain a mixture;
adding yeast to the mixture;
hydrolysing the mixture at a dry matter content of 55% by weight or less, for a time period of from 1 to 48 hours, at a temperature from 20 to 60° C.;
optionally, fully inactivating one or more of the carbohydrase(s); and
isolating the feed or food ingredient.

29. The method according to claim 28, wherein the soy hulls have been disintegrated before being mixed with the one or more carbohydrase(s).

30. The method according to claim 28, wherein the yeast comprises one or more Saccharomyces cerevisiae strains, and is added to the mixture before the hydrolysing step.

31. The method according to claim 28, wherein the yeast comprises one or more yeasts selected from spent brewer's yeast, spent distiller's yeast, spent yeast from wine production, bioethanol yeast, spent yeast from bioethanol production, baker's yeast, and yeast strains fermenting C5 sugars, and is added to the mixture before the hydrolysing step.

32. The method according to claim 28, further comprising adding one or more microorganisms to the mixture before the hydrolysing step.

33. The method according to claim 32, wherein the one or more microorganisms comprise lactic acid bacteria.

34. The method according to claim 28, further comprising adding α-galactosidase to the mixture before the hydrolysing step.

35. A feed, food product, or nutritional supplement comprising from 0.5 to 99% by weight of the feed or food ingredient according to claim 19.

36. A method of feeding a production animal, comprising feeding the production animal a diet comprising the feed, food product, or nutritional supplement of claim 35.

37. The method according to claim 36, wherein the production animal is selected from piglets, calves, and chickens.
